(19) United States
(12) Patent Application Publication
(10) Pub. No.: US 2007/0265520 A1
(43) Pub. Date: Nov. 15, 2007

(54) Magnetic resonance spectroscopy with real-time correction of motion and frequency drift, and real-time shimming

(76) Inventor: Stefan Posse, (US)

Correspondence Address: Stefan Posse, 1616 Bayita Ln NW, Albuquerque, NM, 87107, US

(21) Appl. No.: 11/789,633

(22) Filed: Apr. 24, 2007

Related U.S. Application Data
(60) Provisional application No. 60/795,381, filed on Apr. 27, 2006.

(51) Int. Cl. A61B 5/05

(52) U.S. Cl. 600/410

(57) ABSTRACT

This invention relates to localized magnetic resonance spectroscopy (MRS) and to magnetic resonance spectroscopic imaging (MRSI) of the proton NMR signal, specifically to a magnetic resonance spectroscopy (MRS) method to measure a single volume of interest and to a magnetic resonance spectroscopic imaging method with at least one spectral dimension and up to three spatial dimensions. MRS and MRSI are sensitive to movement of the object to be imaged and to frequency drifts during the scan that may arise from scanner instability, field drift, respiration, and shim coil heating due to gradient switching. Inter-scan and intra-scan movement leads to line broadening and changes in spectral pattern secondary to changes in partial volume effects in localized MRS. In MRSI movement leads to ghosting artifacts across the entire spectroscopic image. For both MRS an MRSI movement changes the magnetic field inhomogeneity, which requires dynamic reshimming. Frequency drifts in MRS and MRSI degrade water suppression, prevent coherent signal averaging over the time course of the scan and interfere with gradient encoding, thus leading to a loss in localization. It is desirable to measure object movement and frequency drift and to correct object motion and frequency drift without interfering with the MRS and MRSI data acquisition.

### REFERENCE TO RELATED APPLICATIONS

Applicant claims priority of U.S. Provisional Application No. 60/795,381, filed on Apr. 27, 2006 for SYSTEM AND METHODS FOR MAGNETIC RESONANCE SPECTROSCOPIC IMAGING WITH SPATIALLY RESOLVED FREQUENCY DRIFT CORRECTION INTEGRATED INTO THE WATER SUPPRESSION MODULE of Stefan Posse, Applicant herein.

### FEDERALLY SPONSORED RESEARCH

The present invention was made with government support under Grant No. 1 R01 DA14178-01 awarded by the National Institutes of Health. As a result, the Government has certain rights in this invention.

### BACKGROUND OF THE INVENTION

1. Technical Field of the Invention

This invention relates to localized magnetic resonance spectroscopy (MRS) and to magnetic resonance spectroscopic imaging (MRSI) of the proton NMR signal, specifically to a magnetic resonance spectroscopy (MRS) method to measure a single volume of interest and to a magnetic resonance spectroscopic imaging method with at least one spectral dimension and up to three spatial dimensions. MRS and MRSI are sensitive to movement of the object to be imaged and to frequency drifts during the scan that may arise from scanner instability, field drift, respiration, and shim coil heating due to gradient switching. Inter-scan and intra-scan movement leads to line broadening and changes in spectral pattern secondary to changes in partial volume effects in localized MRS. In MRSI movement leads to ghosting artifacts across the entire spectroscopic image. For both MRS an MRSI movement changes the magnetic field inhomogeneity, which requires dynamic reshimming. Frequency drifts in MRS and MRSI degrade water suppression, prevent coherent signal averaging over the time course of the scan and interfere with gradient encoding, thus leading to a loss in localization. It is desirable to measure object movement and frequency drift and to correct object motion and frequency drift without interfering with the MRS and MRSI data acquisition.

2. Description of the Prior Art

High-Speed MR Spectroscopic Imaging:

High speed MRSI integrates spatial encoding modules into the spectral acquisition. We have developed Proton-Echo-Planar-Spectroscopic-Imaging (PEPSI) which employs echo-planar readout gradients to accelerate spatial encoding times by more than one order of magnitude as compared to conventional techniques to measure 2-dimensional metabolite distributions at short TE and 3-dimensional metabolite distributions (1,2). PEPSI has also been employed for time-resolved metabolic imaging to dynamically map lactate concentrations during respiratory and metabolic challenges (3,4), to characterize metabolic dysfunction during sodium-lactate infusion in patients with panic disorder (5) and to map multiplet resonance in human brain at short echo time and high field strength (6). We have further increased the encoding speed of high-speed MRSI by combining Proton-Echo-Planar-Spectroscopic-Imaging (PEPSI) with parallel imaging to obtain up to 4-fold acceleration and measurement times of 16 s for a 32×32 matrix with TR 2 s (7) on a 4 Tesla scanner. This technology is particularly advantageous for 3-dimensional spatial mapping and further improvement in encoding efficiency enabled single-shot MRSI (8) in our laboratory.

Motion Detection and Correction

The first on-line prospective real-time methods used straight-line navigators to detect linear motion of organs in the chest (9). These techniques are not applicable in brain scans where rigid body motion in any arbitrary plane and along any axis is possible. In a series of papers, researchers at the Mayo Clinic describe the concepts of orbital and spherical navigators for prospective rigid body motion detection and correction. The orbital (circular) navigator enables the detection of rotation within the plane of the navigator and translations along multiple axes (10, 11). Ward et al. developed a real-time prospective motion correction scheme in which a set of three circular navigators is used to detect motion in all three planes (11). An iterative approach is taken to correct for the motion since the rotations may still be out of the plane of the navigators. The procedure is fairly time consuming and works best for rotations about the cardinal axes. The spherical navigator, first described by Wong and Irarrazabal (12,13) and implemented in a full 3D rigid body measurement application by Welch et al. (14), addresses the problem of off-axis rotations. One implementation of the spherical navigators requires 27 ms for acquisition of the navigator information. Costa et al. have described a 3D rigid body motion correction method using polar spherical navigators (15). By pre-rotating the baseline trajectory of the navigator, the iterations are avoided.

There are no published methods to detect and correct object movement during an ongoing MRS or MRSI scan. Motion correction can be applied post-acquisition using standard registration tools, but the low resolution of the MRSI scan limits the performance of this approach. It is possible to interleave volumetric high resolution MRI scans into the MRS or MRSI scan to detect and prospectively correct movement, but this approach requires additional signal excitation, which interferes with the signal excitation for the spectroscopic acquisition, leading to reduced sensitivity and possible instability in the MRS and MRSI data, and it reduces the temporal resolution of MRS and MRSI.

Compensation of Magnetic Field Inhomogeneity

Magnetic resonance spectroscopic imaging and localized spectroscopy in vivo suffer from microscopic and macroscopic magnetic field inhomogeneity that broaden spectral lines, reduce sensitivity and impair spectral fitting. This is one of the major limitations of MRS and MRSI in vivo. Conventional means of compensating such inhomogeneity include: (a) shimming, which is limited to low shim coils with spatial frequencies and therefore not very effective over large volumes (b) separate acquisition of multiple volumes with different shim settings, which is time consuming (c) increasing spatial resolution, which is very costly in terms of sensitivity and increases measurement time.

Inhomogeneity of the static magnetic field (B₀) can be as large as 6 parts-per-million (ppm) across the brain (16,17). These spatial nonlinearities of local gradients are an important limiting factor in volumetric MRSI studies. Higher order auto-shimming (HOAS) provided on most high-field scanners offers limited capability for correction of such imperfections. While all MR processes will benefit from improved shimming to some degree, specific regions of clinical interest, such as the frontal and medial-temporal brain regions, and acquisition techniques, such as MRSI, can be critically affected by shimming effectiveness. In the case of MRS and MRSI, shim state can adversely affect spectral line width, causing artifactual frequency shifts between voxels and decrease effectiveness of water suppression. Furthermore, poorly suppressed water signal can alias into regions of otherwise adequate water suppression as a result of subject motion or k-space undersampling, causing baseline artifact. Aliasing of residual water signals from regions outside of the volume of interest is particularly difficult to identify.

HOAS typically uses a collection of shim coils based on spherical harmonics or other spatial shapes (for a review, see (18)). These coils are powered by current-feedback amplifiers under the control of a user-addressable interface and analysis program. The corrective fields generated by the coils are of finite number, power and extent. Due to time constraints, HOAS attempts to converge to an optimum shim state analytically rather than iteratively, using field maps collected with the existing imaging capability (19-23). Progress in improving existing technique has focused on addressing the limits of the shimming hardware (24-29) and accuracy and stability of the analysis (30,31). However, for MRS and MRSI the performance of HOAS is still insufficient, in particular at high field.

To overcome large local disturbances in field homogeneity, several methods for correction have been proposed. The use of additional passive ferromagnetic shims in a cylindrical array, placed in close proximity to the human head, has been demonstrated (24). Mouthpieces containing diamagnetic shim material (passive shims) have been developed to enhance the B₀ homogeneity of the mesioinferior frontal lobes (25,26). Hsu and Glover (27) have taken a similar approach but have used a mouthpiece than contains an active shim coil. However, for clinical applications of spectroscopic imaging these approaches are not practical.

Extending the capability of the existing field coil design requires either more coils of higher order (28), or better control over the existing coils. To increase control, Blamire and colleagues (32) showed that a dynamic shim state, following the current acquisition slice, can improve the corrective power of the shim coils by reducing the spatial constraints on the shim state. Subsequent studies have further demonstrated its effectiveness (33). Dynamic shimming offers greater flexibility in compensating local magnetic field distortion, but applications are currently limited by the considerable hardware demands. However, the clinical manufacturers have identified dynamically switched higher order shims as an important advance and have started product development. It is thus foreseeable that switching higher order shims will become clinical routine.

Frequency Drift Correction

MRS and MRSI are sensitive to frequency drifts during the scan that may arise from scanner instability, field drift, respiration, and shim coil heating due to gradient switching. Frequency drifts degrade water suppression, prevent coherent signal averaging over the time course of the scan and interfere with gradient encoding, thus leading to a loss in localization (34-36). Ebel et al. published a method that collects an additional MRSI data set interleaved into the conventional MRSI data acquisition to detect and correct frequency drifts (36). However, this approach is associated with additional signal excitation, which interferes with the signal excitation for the spectroscopic acquisition, leading to reduced sensitivity and possible instability in the MRSI data, and it reduces the temporal resolution of MRSI. It is desirable to measure this frequency shift and to correct the frequency drift without interfering with the MRSI data acquisition. Ideally, this frequency measurement should be performed in small volumes, since this reduces the effect of magnetic field inhomogeneity and makes the frequency measurement more precise. In addition, it is desirable to simultaneously measure the frequency drift in multiple volumes, since the frequency drift may vary in space [34], e.g. due to breathing or due to gradient drift.

U.S. Pat. No. 6,552,539 discloses a method of correcting resonance frequency variation and MRI apparatus. A method of correcting a resonance frequency variation and an MRI apparatus both capable of handling all frequency drifts including a frequency drift whose time change is slow, a frequency drift in a slice direction and a frequency drift whose time change is fast. An amount of a resonance frequency variation is measured, the frequency variation is corrected when an amount of the resonance frequency variation is smaller than a threshold value, and the amount of the resonance frequency variation is not stored. On the other hand, when the amount of the resonance frequency variation is not smaller than the threshold value, the amount of the resonance frequency variation is stored and correction operation is made based thereon later. This method is not applicable to MRS and MRSI.

U.S. Pat. No. 5,166,620 discloses an NMR frequency locking circuit. An NMR locking mechanism for use with not only electromagnets, superconducting magnets and permanent magnets, but also with ultrahigh energy product magnets such as neodynium. The circuit utilizes a single conversion superheterodyne receiver with a phase locked loop that forms a locking mechanism that depends upon a variable frequency. The resonant frequency of the nuclei is compared to a variable excitation frequency which is adjusted to maintain a control frequency with one unique value of the control frequency being zero at lock. This method is suitable for compensating drifts of the main magnetic field, but it is not suitable to compensate movement or changes in magnetic field inhomogeneity.

High-speed MR spectroscopic imaging has important applications.

The development of hyperpolarized MRI agents presents both unprecedented opportunities and new technical challenges. In particular, with signal-to-noise ratio (SNR) enhancements on the order of the 10000-fold, dynamic nuclear polarization of metabolically active substrates (e.g., 13C-labeled pyruvate or acetate) theoretically permits in vivo imaging of not only the injected agent, but also downstream metabolic products. This feature of hyperpolarized MR spectroscopy (MRS) provides investigators a unique opportunity to non-invasively monitor critical dynamic metabolic processes in vivo under both normal and pathologic conditions. Important applications include tumor diagnosis and treatment monitoring, as well as assessment of cardiac function. In studies using hyperpolarized samples, the magnetization decays toward its thermal equilibrium value and is not recoverable. Therefore, fast spectroscopic imaging acquisition schemes are important.

A recent study by Golman et al. (37) described real-time metabolic imaging. NMR spectroscopy has until now been the only noninvasive method to gain insight into the fate of pyruvate in the body, but the low NMR sensitivity even at high field strength has only allowed information about steady-state conditions. The medically relevant information about the distribution, localization, and metabolic rate of the substance during the first minute after the injection has not been obtainable. Use of a hyperpolarization technique has enabled 10-15% polarization of 13C1 in up to a 0.3 Mpyruvate solution. i.v. injection of the solution into rats and pigs allows imaging of the distribution of pyruvate and mapping of its major metabolites lactate and alanine within a time frame of 10 s. Hyperpolarized MRS is currently being developed by major manufacturers and expected to be of considerable commercial value.

MR spectroscopic imaging in moving organs, like the heart or the breast, is sensitive to movement artifact that results in blurring of the image. Gating to the heart beat is frequently used to reduce motion artifact, but this reduces data acquisition efficiency. Simultaneous synchronization to respiration may be required to further reduce motion artifacts, which additionally reduces data acquisition efficiency. Gating in the presence of irregular heart beat introduces variability in repetition time that results in non steady-state signal intensity and distortion of the image encoding process. Image registration during post-processing is challenging due to the highly nonlinear movement pattern within the chest. High-speed spectroscopic imaging acquisition schemes considerably reduce motion sensitivity.

MR spectroscopic imaging in organs, like the brain, is sensitive to localized signal fluctuations due to blood pulsation or other physiological movement mechanisms (e.g. CSF movement) that results in blurring of the image. Gating to the rhythm of the physiological fluctuation (e.g. heart beat) can be used to reduce this artifact, but this reduces data acquisition efficiency. Gating in the presence of irregular heart beat introduces variability in repetition time that results in non steady-state signal intensity and distortion of the image encoding process. Therefore, fast spectroscopic imaging acquisition schemes are important.

This method is also applicable to spatial mapping chemical reactions for applications in material science and biology. For example, the spatial evolution of a chemical chain reaction could be observed. Such reactions are typically very fast and fast spectroscopic imaging acquisition schemes are thus important to avoid blurring of the spectroscopic images.

### REFERENCES

1. Posse S, Tedeschi G, Risinger R, Ogg R, Le Bihan D. High speed 1H spectroscopic imaging in human brain by echo planar spatial-spectral encoding. Magn Reson Med 1995;33(1):34-40.

2. Posse, S., Tedeschi, Risinger, G., Ogg, R., Le Bihan, D., High Speed 1H spectroscopic imaging, Magnetic Resonance in Medicine, 33, 34-40, 1995

3. Posse S, Dager S R, Richards T L, Yuan C, Ogg R, Artru A A, Muller-Gartner H W, Hayes C. In vivo measurement of regional brain metabolic response to hyperventilation using magnetic resonance: proton echo planar spectroscopic imaging (PEPSI). Magn Reson Med 1997;37(6):858-865.

4. Dager S R, Layton M E, Strauss W, Richards T L, Heide A, Friedman S D, Artru A A, Hayes C E, Posse S. Human brain metabolic response to caffeine and the effects of tolerance. Am J Psychiatry 1999;156(2):229-237.

5. Dager S R, Friedman S D, Heide A, Layton M E, Richards T, Artru A, Strauss W, Hayes C, Posse S. Two-dimensional proton echo-planar spectroscopic imaging of brain metabolic changes during lactate-induced panic. Arch Gen Psychiatry 1999;56(1):70-77.

6. Posse, S., Otazo, R., Caprihan, A., Bustillo, J., Chen, H., Henry, P.-G., Marjanska, M., Gasparovic, C., Zuo, C., Magnotta, V., Mueller, B., Mullins, P., Renshaw, P., Ugurbil, K., Lim, K. O., Alger, J. R., Proton Echo Planar Spectroscopic Imaging of J-Coupled Resonances in Human Brain at 3 and 4 Tesla, Magn. Reson. Med., in press

7. Lin, F.-H., Tsai, S.-Y., Otazo, R., Caprihan, A., Wald, L. L., Belliveau, J. W., Posse, S., Sensitivity-encoded (SENSE) Proton-Echo-Planar-Spectroscopic-Imaging (PEPSI) in Human Brain, Magn. Reson. Med., in press

8. Posse, S., Otazo, R., Tsai S-Y, Lin F-H., Single-Shot MR Spectroscopic Imaging with Partial Parallel Imaging, Abstract book: International Society for Magnetic Resonance in Medicine, 2007

9. Firmin D, Keegan J. Navigator echoes in cardiac magnetic resonance. J Cardiovasc Magn Reson. 2001;3:183-193

10. Fu Z W, Wang Y, Grimm R C, Rossman P J, Felmlee J P, Riederer S J, Ehman R L. Orbital navigator echoes for motion measurements in magnetic resonance imaging. Magn Reson Med. 1995;34:746-753

11. Ward H A, Riederer S J, Grimm R C, Ehman R L, Felmlee J P, Jack C R, Jr. Prospective multiaxial motion correction for fMRI. Magn Reson Med. 2000;43:459-469

12. Wong S T, Roos M S. A strategy for sampling on a sphere applied to 3d selective RF pulse design. Magn Reson Med. 1994;32:778-784

13. Irarrazabal P, Nishimura D G. Fast three dimensional magnetic resonance imaging. Magn Reson Med. 1995;33:656-662

14. Welch E B, Manduca A, Grimm R C, Ward H A, Jack C R, Jr. Spherical navigator echoes for full 3d rigid body motion measurement in MRI. Magn Reson Med. 2002;47:32-41

15. Welch E B, Manduca A, Grimm R C, Jack C R, Jr. Interscan registration using navigator echoes. Magn Reson Med. 2004;52:1448-1452

16. Li S, Dardzinski B J, Collins C M, Yang Q X, Smith M B. Three-dimensional mapping of the static magnetic field inside the human head. Magn Reson Med. November 1996; 36(5):705-14.

17. Collins C M, Yang B, Yang Q X, Smith M B. Numerical calculations of the static magnetic field in three-dimensional multi-tissue models of the human head. Magn Reson Imaging. June 2002;20(5):413-24.

18. Romeo F, Hoult D. I. Magnet field profiling: analysis and correcting coil design. Magn. Reson. Med. March 1984;1(1):44-65.

19. Prammer M G, Haselgrove, J. C., Shinnar, M., Leigh, J. S. A new approach to automatic shimming. J Magn Reson 1988; 77:40-52.

20. Webb P., Macovski A. “Rapid, fully automatic, arbitrary volume in vivo shimming” Magn Reson Med. 20(1), 113-22 (1991).

21. Schneider E., Glover G. “Rapid in vivo proton shimming” Magn Reson Med. 18(2), 335-47 (1991).

22. Hu, J., Javaid T., Arias-Mendoza F., Liu Z., Mc Namara R., and Brown T. R., “A fast, reliable, automatic shimming procedure using 1H chemical-shift imaging spectroscopy” J Magn Reson. 108, 213-219 (1995)

23. Reese T G, Davis, T. L., Weisskoff, R. M. Automated shimming at 1.5 T using echo planar image frequency maps. J Magn Reson Imaging 1995; 5(6):739-745.

24. Jesmanowicz A., Hyde J. S., Punchard W. F. B, and Starewicz P. M., “Method for shimming a static magnetic field in a local MRI coil”, U.S. Pat. No. 6,294,972 B1, Issued Sep. 25, 2001.

25. Wilson J L, Jezzard P. Utilization of an intra-oral diamagnetic passive shim in functional MRI of the inferior frontal cortex. Magn Reson Med 2003; 50(5):1089-1094.

26. Cusack R, Russell B, Cox S M, De Panfilis C, Schwarzbauer C, Ansorge R. An evaluation of the use of passive shimming to improve frontal sensitivity in fMRI. Neuroimage 2005; 24(1):82-91.

27. Hsu J J, Glover G H. Mitigation of susceptibility-induced signal loss in neuroimaging using localized shim coils. Magn Reson Med 2005; 53(2):243-248.

28. Hillenbrand D F, Lo, K-M, Punchard W F B, Reese T G, Starewicz P M. “High-order shimming: a simulation study of the effectiveness of competing methods, using an established susceptibility model of the human head”, Applied Magnetic Resonance, Appl Magn Reson 29(1): 39-64, 2005.

29. de Graaf R. A., Brown P. B., McIntyre S., Rothman D. L., Nixon T. W. “Dynamic shim updating (DSU) for multislice shim acquisition” Magn. Reson. Med. 49(3), 409-16 (2003)

30. Kim D. H., Adalsteinsson E., Glover G. H., Spielman D. M. “Regularized high-order in vivo shimming” Magn Reson Med. 48(4), 715-22 (2002).

31. Klassen L. M., Menon R. S., “Robust automated shimming technique using arbitrary mapping acquisition parameters (RASTAMAP)” Magn Reson Med. 51(5), 881-7 (2004).

32. Blamire A M, Rothman D L, Nixon T. “Dynamic shim updating: a new approach towards optimized whole brain shimming.” Magn Reson Med. 36(1) 159-65 (1996).

33. Zhao Y, Anderson A W, Gore J C. “Computer simulation studies of the effects of dynamic shimming on susceptibility artifacts in EPI at high field.” J Magn Reson. 173(1) 10-22 (2005).

34. Henry P G, van de Moortele P F, Giacomini E, Nauerth A, Bloch G. Field-frequency locked in vivo proton MRS on a whole-body spectrometer. Magn Reson Med. October 1999;42(4):636-42.

35. Thiel T, Czisch M, Elbel G K, Hennig J. Phase coherent averaging in magnetic resonance spectroscopy using interleaved navigator scans: compensation of motion artifacts and magnetic field instabilities. Magn Reson Med. June 2002;47(6):1077-82.

36. Ebel A, Maudsley A A. Detection and correction of frequency instabilities for volumetric 1H echo-planar spectroscopic imaging. Magn Reson Med. February 2005; 53(2):465-9.

37. Golman K, in ‘t Zandt R, Thaning M. Real-time metabolic imaging. Proc Natl Acad Sci USA. Jul. 25, 2006;103(30):11270-5. Epub Jul. 12, 2006.

38. van der Kouwe A J, Benner T, Dale A. Real Real-Time Rigid Body Motion Correction and Shimming Using Cloverleaf Navigators. Magn Reson Med., November 2006; 56(5):1019-32.

### SUMMARY OF THE INVENTION

The innovation consists of a modification of the water suppression module in a proton MRS or MRSI sequence to simultaneously measure and correct the frequency drift, the change in magnetic field inhomogeneity in the volume of interest, the object movement, and to suppress the water signal. The water signal that is excited within the water suppression module originates from the entire object seen by the RF coil and the corresponding water spectrum is broadened by magnetic field inhomogeneity, which reduces sensitivity to measuring small frequency shifts. By inserting between the water suppression RF pulse and the dephasing gradient pulses either phase a sensitive MRI encoding module, or a 1D, 2D or 3D high-speed MRSI encoding module it is possible to measure frequency drift, magnetic field inhomogeneity and object movement. This information is used to dynamically change the synthesizer frequency of the scanner, the shim settings and to shift the encoded k-space. In the preferred implementation this information is computed in real-time during the ongoing scan and via feeback loop downloaded to the acquisition control unit to update the aforementioned parameters before the subsequent data acquisition. In the most basic implementation of the method a train of cyclically inverted readout gradients is applied immediately after the water suppression RF pulse to measure and spatially encode the decaying water signal. This train of readout gradients simultaneously encodes one spatial direction and spectral information. Crusher gradients are applied at the end of the readout gradient train to dephase the residual water signal.

It is an object of the present invention to enable magnetic resonance spectroscopic imaging with real-time motion and frequency drift correction.

It is another object of the present invention to provide an magnetic resonance spectroscopic imaging apparatus with real-time motion and frequency drift correction.

These and significant other advantages of the present invention will become clear to those skilled in this art by careful study of this description, accompanying drawings and claims.

### BRIEF DESCRIPTION OF THE DRAWINGS

FIG. 1 shows a PEPSI pulse sequence with water suppression (WS), outer volume suppression (OVS), the spin echo excitation module and the echo-planar readout module. Data are collected in blocks during each of the cyclically inverted readout gradients (Gᵣ).

FIG. 2 shows examples of the integration of data encoding modules into the water suppression module: (a) an MRSI readout module used in the PEPSI sequence, (b) a cloverleaf navigator module developed by van der Kouwe ( ).

FIG. 3 illustrates examples of encoding modules but not limited to these: (a) ID encoding (b) 2D single-shot encoding (c) 3D single shot encoding.

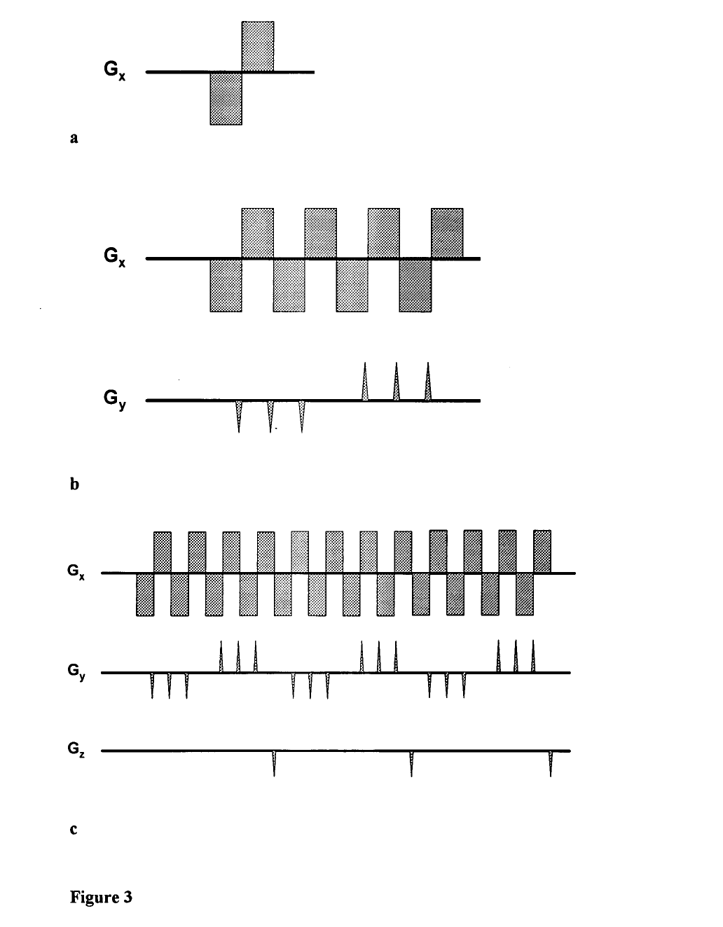

FIG. 4 shows a flow chart of the feedback loop to dynamically change the frequency of the RF subsystem, the shim settings and the magnetic field gradient amplitudes and orientations to change the orientation of the k-space grid during the ongoing scan.

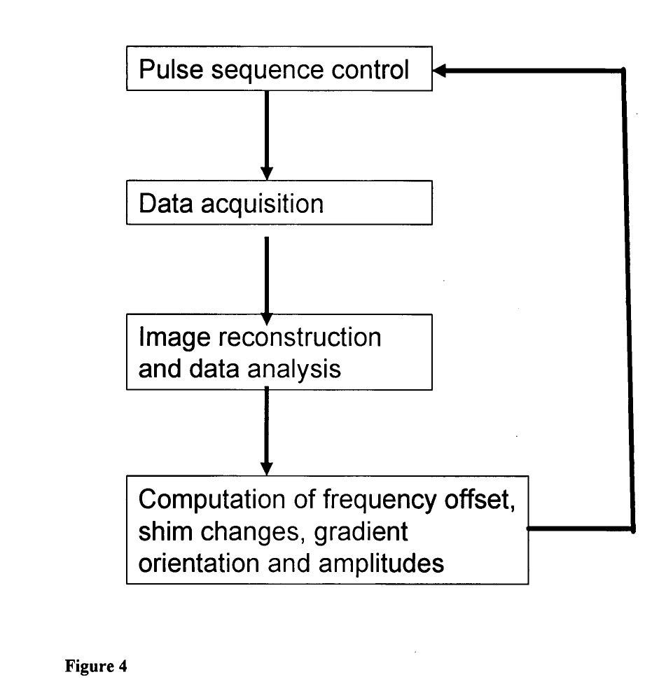

### DETAILED DESCRIPTION OF THE PRESENT INVENTION

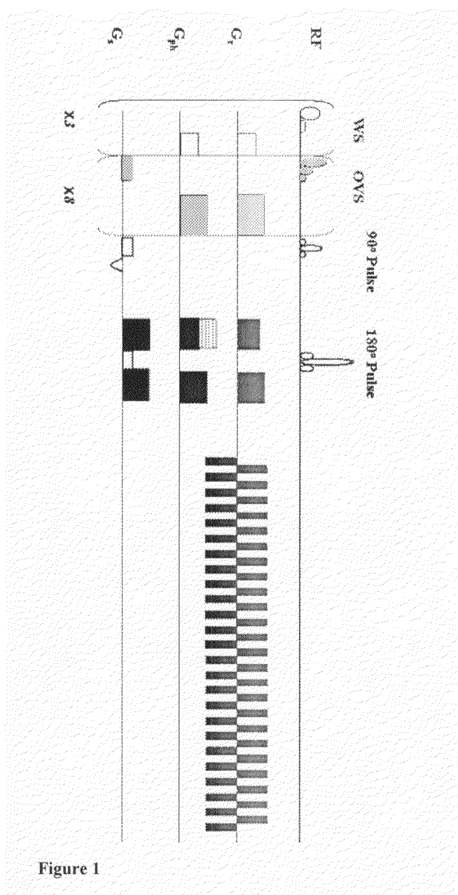

The water signal is deliberately suppressed in localized proton MRS and proton MRSI. This invention spatially-spectrally encodes this signal, which is available immediately after the water excitation pulse, before dephasing it. In this location, the magnetization will be unaffected, and the impact on water suppression and pulse sequence timing will be minimal. FIG. 1 shows a PEPSI pulse sequence (1,2) with water suppression (WS), outer volume suppression (OVS), the spin echo excitation module and the echo-planar readout module. Data are collected in blocks during each of the cyclically inverted readout gradients (Gᵣ). The invention involves the insertion of a spatial encoding module into a single or o multiple water suppression (WS) modules. This modification is not specific to the PEPSI pulse sequence, but can be applied to any localized spectroscopy and spectroscopic imaging pulse sequence that contains water suppression modules. Furthermore, two sets of navigators could be collected in quick succession in consecutive water suppression modules to estimate intra-scan frequency drifts, intra-scan changes in magnetic field inhomogeneity and intra-scan movement.

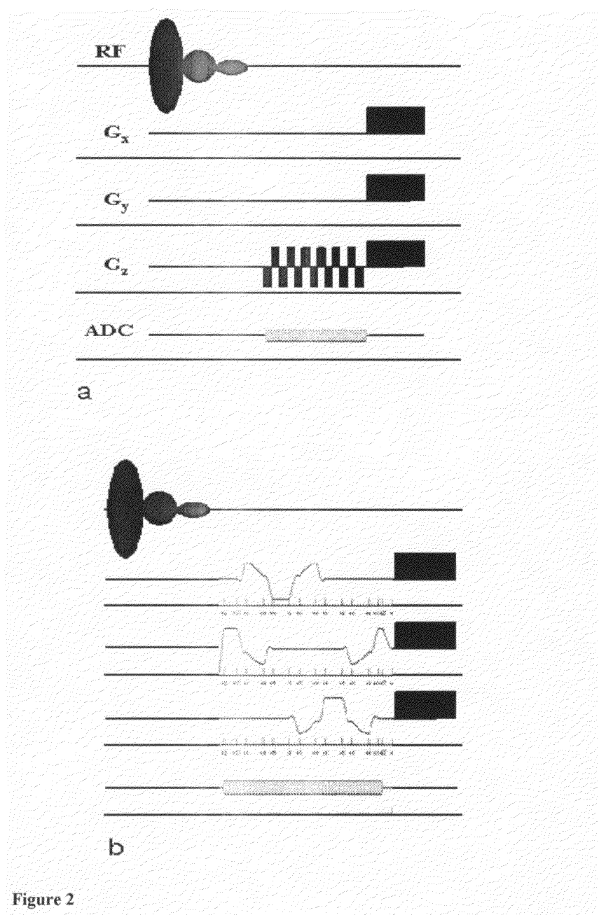

FIG. 2 shows examples of the implementation of data encoding modules into the water suppression. FIG. 2a shows a 1-dimensional spatial encoding module based on the PEPSI sequence. The duration of the readout gradient train determines the spectral resolution. It is limited by the total duration of the water suppression module and the duration of the water suppression RF pulse, which cannot be changed. The duration of each individual readout gradient determines the spectral width (1,2). Repetition of this measurement in consecutive water suppression modules enables extrapolation of intra-scan frequency drifts, shim changes and movement.

In the human brain magnetic field inhomogeneities are most prominent along the z-direction. Using a z-gradient to spatially resolve the magnetic field inhomogeneities will thus improve the detection of small frequency shifts that may vary between individual axial slices. In general the readout module should be applied along the direction with greatest magnetic field inhomogeneity. Further improvement in the precision of the frequency measurement can be achieved by encoding 2D or 3D space, simultaneously with spectral information to obtain column- or voxel-resolved frequency information. These 2D and 3D encoding modules are either applied in a single shot and repeated rapidly to encode spectral information, or applied across multiple acquisition steps of the main MRSI sequence.

FIG. 2b shows the cloverleaf navigator, which has been designed and tested for use in gradient echo imaging sequences by van der Kouwe et al (38). Every navigator scan will not be in the steady-state as in the FLASH sequence. The non steady-state behavior of the navigators across the train of echoes can be modeled, mapped and corrected. Early experience with a conventional MRSI sequence suggests that even without correction, reasonable though biased estimates result. The bias will be reduced by correcting for the non-steady-state. Second, if the rotation estimates are biased, averaging will not reduce the bias. If this appears to be a problem, we will lengthen the arc segments of the navigators (e.g. to 180 degrees) and/or include navigators with different radii in the train (thus interrogating the object at different resolutions and improving the rotation estimates). The accuracy of the translation estimates from the navigators may also be compromised by field drifts. Zeroth order offsets in the B0 field appear as a linear phase roll across the navigator readout that is independent of the applied gradient. Improved phase estimation will be obtained by using multiple repetitions of the cloverleaf navigator within a water suppression module. We have observed that instantaneous frequency estimates from cloverleaf navigators embedded in gradient echo imaging sequences have a noise component with a variance of less than 1 Hz (38). Since several repeated estimates will be made at regular intervals in the spectroscopy sequence, and assuming the drift estimates are unbiased, the accuracy can be increased by low-pass filtering to well below the target resolution of 1 Hz.

Magnetic field inhomogeneity information can be collected using more fully encoded 2D or 3D spatial MRSI modules, or 3D or 3D phase sensitive MRI modules.

The frequency drift correction, required shim changes to compensate motion related (or due to other factors) changes in magnetic field inhomogeneity and required changes in the magnetic field gradient orientation to compensate movement will be computed online and downloaded into the pulse sequence to enable online correction of all transmit RF pulse frequencies, gradient amplitudes and orientations and the receiver frequency offset. Most clinical scanners provide a convenient mechanism (e.g. used in the Siemens PACE motion correction implementation in the product sequences (1) for rapidly feeding back corrections to the gradient and RF subsystems in a closed-loop time of less than 5 ms. On a Siemens scanner this can be carried out using the Image Calculation Environment (postprocessing software) of the scanner. Online movement correction may be performed using the product PACE method implemented in the ICE reconstruction program (38).

What is claimed is:

1. An MRI apparatus that permits collecting a complete spectroscopic image with one spectral dimension and up to three spatial dimensions in a single signal excitation comprising:
an RF pulse transmitting device to excite nuclear spins in a circumscribed region;
a gradient pulse application device to encode k-space;
an NMR signal receiving device;
a spatial-spectral data collection, reconstruction and storage device; and
a pulse sequence control device to generate a magnetic resonance spectroscopy pulse sequence and a magnetic resonance spectroscopic imaging pulse sequence containing a modified water suppression module.

2. An MRI apparatus with a pulse sequence control device according to claim 1, further comprising: inserting an at least 1-dimensional encoding module between the radiofrequency excitation pulse and the first dephasing gradient pulse of the water suppression module.

3. An MRI apparatus with a pulse sequence control device according to claim 1, further comprising: real-time motion correction using an at least 1-dimensional spatial encoding module within the modified water suppression module that uses magnetic field gradients.

4. An MRI apparatus with a pulse sequence control device according to claim 1, further comprising: real-time motion correction using an at least 1-dimensional spatial encoding module within the modified water suppression module that uses partial parallel imaging with radiofrequency array coils.

5. An MRI apparatus with a pulse sequence control device according to claim 1, further comprising: real-time motion correction using an at least 1-dimensional spatial encoding module within the modified water suppression module that uses magnetic field gradients and partial parallel imaging with radiofrequency array coils.

6. An MRI apparatus with a pulse sequence control device according to claim 1, further comprising: real-time frequency drift correction using an at least 1-dimensional spatial encoding module within the modified water suppression module that uses magnetic field gradients.

7. An MRI apparatus with a pulse sequence control device according to claim 1, further comprising: real-time frequency drift correction using an at least 1-dimensional spatial encoding module within the modified water suppression module that uses partial parallel imaging with radiofrequency array coils.

8. An MRI apparatus with a pulse sequence control device according to claim 1, further comprising: real-time frequency drift correction using an at least 1-dimensional spatial encoding module within the modified water suppression module that uses magnetic field gradients and partial parallel imaging with radiofrequency array coils.

9. An MRI apparatus with a pulse sequence control device according to claim 1, further comprising: real-time magnetic field inhomogeneity correction using an at least 1-dimensional spatial encoding module within the modified water suppression module that uses magnetic field gradients.

10. An MRI apparatus with a pulse sequence control device according to claim 1, further comprising: real-time magnetic field inhomogeneity correction using an at least 1-dimensional spatial encoding module within the modified water suppression module that uses partial parallel imaging with radiofrequency array coils.

11. An MRI apparatus with a pulse sequence control device according to claim 1, further comprising: real-time magnetic field inhomogeneity correction using an at least 1-dimensional spatial encoding module within the modified water suppression module that uses magnetic field gradients and partial parallel imaging with radiofrequency array coils.

12. An MRI apparatus with a pulse sequence control device according to claim 1, further comprising: one or more repetitions of the modified water suppression module.

13. An MRI apparatus with a pulse sequence control device according to claim 1, further comprising: a real-time data analysis device, a real-time decision device and a feedback device to modulate the pulse sequence control device.

14. An MRI apparatus that permits collecting a complete spectroscopic image with one spectral dimension and up to three spatial dimensions in a single signal excitation comprising:
an RF pulse transmitting device to excite nuclear spins in a circumscribed region;
a gradient pulse application device to encode k-space;
an NMR signal receiving device;
a spatial-spectral data collection, reconstruction and storage device; and
a pulse sequence control device to generate a magnetic resonance spectroscopy pulse sequence and a magnetic resonance spectroscopic imaging pulse sequence containing a real-time data analysis device and a feedback device to modulate the pulse sequence control device.

15. A magnetic resonance spectroscopic imaging apparatus with real-time motion and frequency drift correction, and real-time shimming according to claim 14, further comprising: a device to measure and compare movement of the object, frequency drift of the acquired signal and magnetic field inhomogeneity in the object of the currently acquired data with the acquired data in the data previous repetition of the pulse sequence.

16. A magnetic resonance spectroscopic imaging apparatus with real-time motion and frequency drift correction, and real-time shimming according to claim 14, further comprising: a decision device to determine the change in RF subsystem frequency, gradient subsystem amplitudes and orientation, and shim settings.

17. A magnetic resonance spectroscopic imaging apparatus with real-time motion and frequency drift correction, and real-time shimming according to claim 14, further comprising: a real-time feedback-loop to accomplish the correction of the RF subsystem frequency, gradient subsystem amplitudes and orientation, and shim settings.

18. A method of magnetic resonance spectroscopic imaging with real-time motion and frequency drift correction comprising the steps of: providing a cloverleaf navigator designed and tested for use in gradient echo imaging sequences; modifying the behavior navigators across the train of echoes water suppression; modifying modules to include a short navigator immediately after the RF pulse; and providing a dephasing gradient.

19. A method of magnetic resonance spectroscopic imaging with real-time motion and frequency drift correction according to claim 18, further comprising the step of: obtaining improved phase estimation by using multiple repetitions of the cloverleaf navigator within a water suppression module.

20. A method of magnetic resonance spectroscopic imaging with real-time motion and frequency drift correction according to claim 18, further comprising the step of: increasing the accuracy by using low-pass filtering to well below the target resolution of 1 Hz.
